(19) United States
(12) Patent Application Publication
(10) Pub. No.: US 2020/0365253 A1
(43) Pub. Date: Nov. 19, 2020

(54) SERVER FOR BUILDING BIG DATA DATABASE BASED ON QUANTIFICATION AND ANALYSIS OF MEDICAL IMAGES AND SERVER-BASED MEDICAL IMAGE ANALYSIS METHOD

(71) Applicants: Coreline Soft Co., Ltd., Seoul (KR); UNIVERSITY OF ULSAN FOUNDATION FOR INDUSTRY COOPERATION, Ulsan (KR); The Asan Foundation, Seoul (KR)

(72) Inventors: Jaeyoun YI, Seoul (KR); Byeongsoo KIM, Bucheon-si, Gyeonggi-do (KR); Hyeon JI, Seoul (KR); Donghoon YU, Gimpo-si (KR); Yongjin CHANG, Incheon (KR); Joon Beom SEO, Seoul (KR); Sang Min LEE, Seoul (KR); Namkug KIM, Seoul (KR)

(21) Appl. No.: 15/931,083

(22) Filed: May 13, 2020

(30) Foreign Application Priority Data
May 14, 2019 (KR) 10-2019-0056152

(51) Int. Cl. G16H 30/40; G06T 7/00; G06K 9/46

(52) U.S. Cl. CPC G16H 30/40; G06T 2207/10081; G06K 9/46; G06T 7/0014

(57) ABSTRACT

Disclosed are a server and a server-based medical image analysis method. A medical image analysis server according to an embodiment of the present invention includes at least one processor. The at least one processor is configured to: automatically transmit a retrieval query to a first database in which medical images are stored; control a receiving interface so that the receiving interface receives a first medical image satisfying the retrieval query from the first database; perform image processing on the first medical image and extract at least one first region of interest from the first medical image; quantify a first feature extracted for the first medical image and the at least one first region of interest; and store the first feature in a second database in association with the first medical image and the retrieve condition.

### CROSS-REFERENCE TO RELATED APPLICATION

This application claims under 35 U.S.C. § 119(a) the benefit of Korean Patent Application No. 10-2019-0056152 filed on May 14, 2019, which is incorporated herein by reference.

### TECHNICAL FIELD

The present invention relates generally to a server and a server-based medical image analysis method, and more particularly to technology for efficiently building a big data database of medical images based on the analysis and quantification of the medical images.

### BACKGROUND ART

A clinical decision support system is a system that provides doctors with assistance with decision making by providing the function of providing required base knowledge and also helping correct reasoning when doctors make diagnoses or determine treatment policies in the treatment of patients. In the examination of a patient, in addition to the subjective decision of a doctor in charge, medically established guidelines are implemented using a computer and then the doctor is informed of the results of the guidelines on the patient's condition, thereby preventing misdiagnosis and also enabling more objective medical practice.

Korean Patent No. 10-1744800 entitled “System for Providing Medical Information” introduces a technique for extracting similar cases by applying an analytic hierarchy process (AHP) based on weights and also comparing the attribute information of a patient and the already stored case information of patients, into which the weights have been incorporated, in order to extract cases similar to that of the patient.

However, even according to this related art, the already stored case information of patients includes only diagnostic information input by doctors, and the analysis of the stored data may be analysis without clinical meaning. Typically, it is vulnerable to problems such as overfit, and the reliability of the analysis is particularly low when there is an insufficient amount of data.

Recently, with the development of artificial intelligence technology represented by machine learning based on an artificial neural network, various techniques for processing big data have been developed, and attempts have been actively made to assist in clinical decision making by applying artificial intelligence to medical information. In particular, there have been developed methods of helping clinicians make decisions by applying artificial intelligence algorithms not only to medical images acquired from diagnostic apparatuses such as an X-ray machine, an ultrasonic scanner, a computed tomography (CT) scanner, a magnetic resonance imaging (MRI) scanner, a positron emission tomography (PET) scanner, etc. but also to various types of medical information including medical histories, health-related numerical data, etc.

Attempts to process big data by applying artificial intelligence to medical information include Korean Patent No. 10-1884609 entitled “System for Diagnosing Disease through Modularized Reinforcement Learning.” However, even according to this preceding literature, the focus is placed only on the classification and pattern extraction of unstructured data, and it is not clear whether or not an extracted pattern is clinically meaningful. Accordingly, this technique is not suitable for practical application in the medical field.

U.S. Pat. No. 10,248,759 entitled “Medical Imaging Reference Retrieval and Report Generation” is a preceding document in which a user is assumed to be a clinician or radiologist in order to acquire clinically meaningful data when retrieving similar cases. This preceding document discloses a technique for automatically retrieving images including a certain feature, receiving feedback (selection), regarding whether or not the corresponding images are suitable for the results of the search, from a user, and generating a report.

U.S. Pat. No. 7,724,930 entitled “Systems and Methods for Automatic Change Quantification for Medical Decision Support” provides a means for comparing a patient's previous medical image with his or her current medical image, automatically quantifying changes in a specific area, and then generating a report.

Even according to the above-described preceding documents, the following problems still exist. First, the lack of clinically meaningful data remains unresolved. Second, due to analysis based on limited data, an erroneous pattern is acquired because of data overfit or a clinically meaningless pattern is acquired.

### SUMMARY

The present invention has been conceived to overcome the above-described problems, and an object of the present invention is to generate a large amount of clinically meaningful data by adding the workflow of the present invention to a workflow for processing medical information inside a medical institution.

An object of the present invention is to generate a large amount of clinically meaningful data based on medical information inside a medical institution without invading patients' privacy.

An object of the present invention is to enrich a medical information database and build a big data database.

An object of the present invention is to provide a medical image analysis technique capable of effectively supporting search for similar cases and presenting more cases and a larger amount of information when searching for similar cases.

An object of the present invention is to provide a medical image analysis technique capable of building a big data database in a server-based environment.

In accordance with an aspect of the present invention, there is provided a medical image analysis method performed by a server, the medical image analysis method including: automatically retrieving a medical image satisfying/corresponding to a retrieve condition in a first database in which medical images are stored, and receiving a retrieved image as a first medical image; performing image processing on the first medical image, and extracting at least one first region of interest from the first medical image; quantifying a first feature extracted for the first medical image and the at least one first region of interest; and storing the first feature in a second database in association with the first medical image and the retrieve condition.

The medical image analysis method may further include: incorporating the first feature into a data set related to the retrieve condition; generating statistical information about the data set related to the retrieve condition; and storing the statistical information in the second database in association with the retrieve condition.

The medical image analysis method may further include: receiving a new medical image of a patient as a second medical image; performing image processing on the second medical image, and extracting at least one second region of interest from the second medical image; quantifying a second feature extracted for the second medical image and the at least one second region of interest; and generating results, obtained by comparing the second feature with the statistical information, as a diagnostic report for the second medical image.

The medical image analysis method may further include: generating a quantitative retrieve condition for a quantified feature based on the second feature; and retrieving a medical image satisfying/corresponding to the quantitative retrieve condition and the retrieve condition in the first database, and providing a retrieved image to the user as a fourth medical image. In this case, the quantitative retrieve condition may be set such that an image having an analysis value similar to the analysis value of a current image of the patient is to be searched for.

The medical image analysis method may further include: providing a user with a user menu adapted to add a quantitative retrieve condition for a quantified feature to the retrieve condition; determining the quantitative retrieve condition based on the user's input via the user menu; and retrieving a medical image satisfying/corresponding to the quantitative retrieve condition and the retrieve condition in the first database, and providing a retrieved image to the user as a third medical image.

The medical image analysis method may further include: generating results, obtained by comparing the first feature with the statistical information, as a diagnostic report for the first medical image.

The medical image analysis method may further include: generating label information for the first medical image based on the diagnostic report for the first medical image; and generating a fifth medical image by adding the label information to the first medical image.

The type of the at least one first region of interest, the category of the first feature, and an image processing process for the first medical image may be determined based on the retrieve condition in advance. The retrieve condition may be predetermined to include at least one of the type and feature of the at least one first region of interest, the category of the first feature, and an image processing process for the first medical image in a common fashion.

In accordance with another aspect of the present invention, there is provided a medical image analysis server including at least one processor, wherein the at least one processor is configured to: automatically transmit a retrieval query to a first database in which medical images are stored; control a receiving interface so that the receiving interface receives a first medical image satisfying/corresponding to the retrieval query from the first database; perform image processing on the first medical image and extract at least one first region of interest from the first medical image; quantify a first feature extracted for the first medical image and the at least one first region of interest; and store the first feature in a second database in association with the first medical image and the retrieve condition.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a diagram showing a thin client environment including a server according to an embodiment of the present invention; and

FIG. 2 is an operation flowchart showing an example of an image analysis method that is performed by the server of FIG. 1.

### DETAILED DESCRIPTION

Other objects and features of the present invention in addition to the above objects will be apparent from the following description of embodiments taken in conjunction with the accompanying drawings.

Embodiments of the present invention will be described in detail below with reference to the accompanying drawings. In the following description of the present invention, when it is determined that a detailed description of a related known component or function may unnecessarily make the gist of the present invention obscure, it will be omitted.

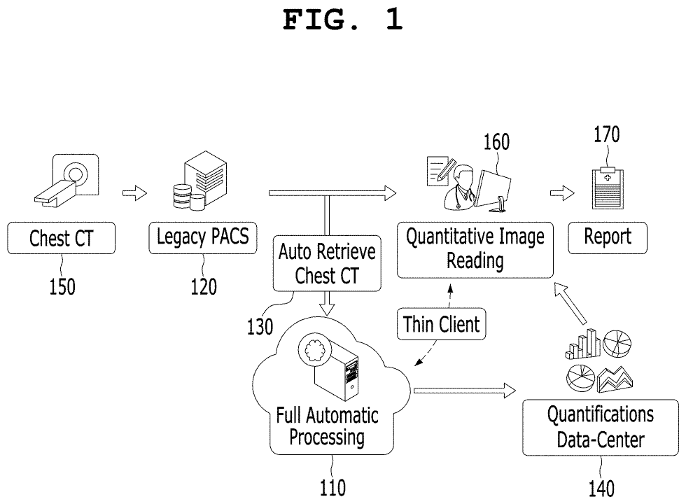

FIG. 1 is a diagram showing a thin client environment including a server according to an embodiment of the present invention.

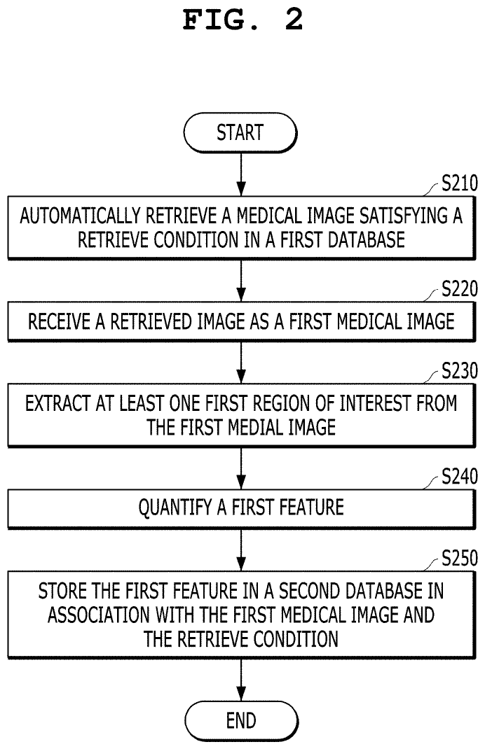

FIG. 2 is an operation flowchart showing an example of an image analysis method that is performed by the server of FIG. 1.

Referring to FIGS. 1 and 2, at step S210, a server 110 automatically searches for a medical image satisfying/corresponding to a “retrieve condition” in a first database 120 in which medical images are stored. Step S210 may be performed in such a manner that the server 110 automatically transmits a retrieval query including the retrieve condition to the first database 120.

Although not shown in FIG. 1, the server 110 may include at least one processor. In this case, the medical image analysis method shown in FIG. 2 may be implemented in the form of computer-executable program instructions. The medical image analysis method shown in FIG. 2 may be performed in such a manner that the program instructions are stored in memory or caches and loaded into and performed by the at least one processor of the server 110.

The server 110 may automatically transmit a retrieval query based on the retrieve condition to the first database 120 according to a routine inside the server 110 without either a request/command made by a user or an instruction received from the outside. In this case, the server 110 may periodically or aperiodically transmit the retrieval query to the first database 120. In the aperiodic case, for example, when the amount of medical image data stored in the first database 120 after a previous search based on a specific retrieve condition is equal to or larger than a predetermined amount, the server 110 may generate a query about the specific retrieve condition, and may retrieve a medical image.

The at least one processor of the server 110 may control a receiving interface/receiving module/receiver (not shown) inside the server 110 so that the receiving interface receives a first medical image satisfying/corresponding to the retrieval query/retrieve condition from the first database 120.

A medical image acquired by an imaging modality 150 may be stored in the first database 120, and may then be transmitted from the first database 120 to a user terminal in response to a request from a clinician or radiologist.

In this case, although a chest CT scanner is shown as the modality 150 in FIG. 1, this is merely an embodiment of the present invention, and one or more of a plurality of diagnostic imaging apparatuses, such as an X-ray machine, a CT scanner, an MRI scanner, a PET-CT scanner, a fluoroscope, an ultrasonic diagnostic imaging apparatus, etc., may be selected as the modality 150.

The first database 120 is a general database configured to store medical images, and may be, e.g., a legacy Picture Archive and Communication System (PACS), as shown in FIG. 1. According to another embodiment of the present invention, the first database 120 may be a database that is held by the server.

At step S220, the server 110 receives a retrieved image as the first medical image. This step is illustrated as an example in conjunction with an automatically retrieved chest CT image 130 in FIG. 1. The retrieve condition may be, e.g., a CT image including the chest, as shown in FIG. 1, and a diagnosis target at the step of quantitative image reading 160 is, e.g., chronic obstructive pulmonary disease (COPD). It will be apparent to those skilled in the art that the spirit of the present invention is not limited to these embodiments.

The server 110 performs image processing on the first medical image. At step S230, the server 110 extracts at least one first region of interest from the first medical image as a result of the image processing. The image processing step may include one or more image processing steps.

At step S240, the server 110 extracts a first feature for the first medical image and the at least one first region of interest and quantifies the first feature.

At step S250, the server 110 stores the first feature in a second database 140 in association with the first medical image and the retrieve condition.

The second database 140 is a database configured to store the quantified first feature and the search result for the retrieve condition in association with each other, and is operated under the management of the server 110.

The medical image analysis method performed by the server 110 of the present invention may further include the steps of: incorporating the first feature into a data set related to the retrieve condition; generating statistical information about the data set related to the retrieve condition; and storing the statistical information in the second database in association with the retrieve condition. The generation of the statistical information is performed by the server 110, and may be performed in a thin-client environment. In other words, the server 110 may automatically retrieve and analyze medical images satisfying a specific retrieve condition among the medical images stored in the first database 120 without consuming processing power at the user terminal. In an embodiment, this step may be performed in the form of a background operation.

The functions that may be provided by the server 110 of the present invention in connection with the quantitative image reading 160 that is performed in a user terminal used by a user (a clinician or radiologist) will be described below. The user terminal in which the quantitative image reading 160 is performed is connected to the server 110 over a wired or wireless connection, and there is supported a thin client environment in which the performance of the computation requested by the user terminal is led by the server 110.

When a user receives a new medical image of a specific patient, the user may request support for the quantitative image reading 160 of the medical image from the server 110. The user may receive a second medical image, which is a new medical image of the patient, from the modality 150 or the first database 120.

In this case, the medical image analysis method performed by the server 110 of the present invention may further include the steps of: receiving a second medical image, which is a new medical image of the patient, from the first database 120; performing image processing on the second medical image, and extracting at least one second region of interest from the second medical image; quantifying a second feature extracted for the second medical image and the at least one second region of interest; and generating results, obtained by comparing the second feature with the statistical information, as a diagnostic report 170 for the second medical image. In this case, the server 110 may provide a user terminal with a means for quantifying and evaluating the severity of the patient's symptoms shown in the second medical image as a function of supporting the quantitative image reading 160. In other words, the distribution of the quantified indices of an overall patient group for a specific disease can be known, and quantified information about a range to which the patient currently belongs to in the overall patient group may be provided to the user terminal.

In this case, the medical image analysis method performed by the server 110 of the present invention may further include the steps of:

generating a quantitative retrieve condition for a quantified feature based on the second feature; and retrieving a medical image satisfying the quantitative retrieve condition and the retrieve condition in the first database, and providing a retrieved image to the user as a fourth medical image. The quantitative retrieve condition may be set such that an image having an analysis value similar to the analysis value of the patient's current image is to be searched for. The server 110 may provide a quantitative retrieve condition adapted to retrieve similar cases having a quantitative feature (within the same category as the second feature) similar to that of the second medical image, which is a current image of the patient, and may increase the efficiency of retrieve similar cases requested by the user from the user terminal as the function of supporting the quantitative image reading 160.

The medical image analysis method performed by the server 110 according to an embodiment of the present invention may further include the steps of: providing a user with a user menu adapted to add a quantitative retrieve condition for a quantified feature to the retrieve condition; determining the quantitative retrieve condition based on the user's input via the user menu; and retrieving a medical image satisfying the quantitative retrieve condition and the retrieve condition in the first database, and providing a retrieved image to the user as a third medical image. In this case, unlike in the previous embodiment, the server 110 may provide the user terminal with a user menu adapted to add a quantitative condition for the quantified feature to the retrieve condition even when there is no comparison target reference image of the patient as a function of supporting the quantitative image reading 160. In this case, when combined with statistical information, it may be possible to retrieve cases based on the severity of a specific disease according to the quantity retrieve condition, thereby retrieving typical cases according to their severity and generating reference images required for training for clinicians and radiologists and training for machine learning based on an artificial neural network. In other words, the application of the function of supporting the quantitative image reading 160 may be extended to a wide range including education for users or training for machine learning based on an artificial neural network.

The method for analyzing medical images performed by the server 110 of the present invention may further include the step of generating results, obtained by comparing the first feature with the statistical information, as a diagnostic report 170 for the first medical image.

In this case, the medical image analysis method performed by the server 110 of the present invention may further include the steps of: generating label information for the first medical image based on the diagnostic report 170 for the first medical image; and generating a fifth medical image by adding the label information to the first medical image. When label information is added to each medical image, a reference image required for training for machine learning based on an artificial neural network may be generated.

The type of the at least one first region of interest, the category of the first feature, and an image processing process for the first medical image may be determined based on the retrieve condition in advance. Furthermore, the retrieve condition may be predetermined to include at least one of the type and feature of the at least one first region of interest, the category of the first feature, and the image processing process for the first medical image in a common fashion.

The retrieve condition may be set to have a range wider than a range corresponding to an analysis target and an analysis method in order to include a plurality of analysis targets and a plurality of analysis methods.

For example, the retrieve condition may be set to “a case that is acquired by a CT modality, related to the chest, and includes 200 or more slices so that image analysis can be performed” among medical image data stored in a legacy PACS, which is the first database 120. In this case, since not all the data of the legacy PACS is received, the amount of data may be adjusted realistically (the amount of data received may be adjusted through the setting of the retrieve condition). For example, a separate storage space inside the second database 140 or the server 110 may be implemented using storage such as network-attached storage (NAS).

According to an embodiment of the present invention, the retrieve condition may be set to a case that concerns chest CT as described above and includes 200 or more slices, and the at least one first region of interest may be set to a region where the probability of COPD is higher than a threshold. The image processing of the first medical image adapted to extract the first region of interest may include image segmentation and measurement.

Although the retrieve condition is exemplified as a simple case where a target image concerns chest CT and a diagnosis target is COPD for ease of description, the spirit of the present invention is not limited to this example.

According to another embodiment of the present invention, the server 110 sets retrieve conditions for various lesions and/or diseases and retrieve conditions in connection with various body parts, and automatic search, quantification and medical image analysis are performed for each of the retrieve conditions when a retrieve each retrieve condition is required (e.g., when a predetermined period of time has elapsed since a previous search, or when a predetermined amount of data has been stored in the legacy PACS since a previous search).

The retrieve condition may include information about a modality by which a medical image is acquired. Furthermore, the retrieve condition may include information about whether or not a specific body part of a human body or an organ is included.

The retrieve condition may be set to a relatively comprehensive condition, for example, in order to allow image analysis to be performed on a region where a plurality of lesions or a plurality of types of diseases may occur. For example, the retrieve condition may be set such that all CT images including the chest are searched for in medical images, as described above. Alternatively, a case including 200 or more slices may be added to the retrieve condition in order to enable in-depth analysis. In this case, the chest may include the lungs, the heart, the liver, and/or the like.

The steps of performing image processing on a first medical image and extracting at least one first region of interest, and/or the steps of extracting and quantifying a first feature for the at least one first region of interest may be determined based on the retrieve condition.

For example, a medical image including the chest may include the lungs, the heart, the liver, and/or the like. Accordingly, when image processing is performed on the first medical image, the features that the organs of the human body based on the retrieve condition may have according to modality may be considered. Furthermore, the first region of interest may also be set to organs of the human body that the first medical image may have based on the retrieve condition, and/or to a disease or lesion that specific organs may include.

For example, in the case of a medical image including the lung, a pulmonary nodule may be extracted as the first region of interest, and a region where a possibility of COPD is higher than a threshold value may be extracted as the first region of interest. In other words, when the retrieve condition concerns chest CT, both the detection of a pulmonary nodule and the detection of COPD are performed on the first medical image, and the first region of interest may include a suspected pulmonary nodule or COPD region. In this case, each region of interest may additionally include information indicating whether it corresponds to a pulmonary nodule or COPD.

The step of extracting the first region of interest that is performed on the first medical image automatically retrieved by the server 110 may be also performed on the second medical image that is a current image of the patient and is subjected to the quantitative image reading 160 in response to a request from the user terminal. However, in this case, the quantitative image reading 160 is clearly and frequently performed for the purpose of diagnosing a specific disease, and thus performance may be conducted such that the second region of interest includes a smaller amount of information than the first region of interest. In other words, the extraction of the region of interest that is automatically performed by the server 110 may be performed on both a pulmonary nodule and COPD, and the quantitative image reading 160 that is performed in response to a request from the user terminal may be performed on any one of a pulmonary nodule and COPD.

The server 110 may automatically retrieve an image in a thin client environment without separate user input, may automatically analyze the image, and may automatically perform quantification. This workflow may be implemented by being added to the workflow “the modality 150→the first database 120→the quantitative image reading 160→report generation 170,” which is a workflow performed in an existing medical institution, and does not interfere with an existing workflow.

In this case, an analysis step is automatically performed without user input, so that the inconvenience of manually transmitting medical images is not caused and the possibility of omission attributable to a mistake will be reduced. Furthermore, images are periodically or aperiodically analyzed and also automatic image search and analysis are performed in the case where a predetermined period of time has elapsed or a predetermined quantity of medical images has been stored in the first database 120 since a previous search, so that a medical image matching the retrieve condition may be prevented from being omitted.

As described above, the method of analyzing medical images based on the server 110 according to the present invention may secure a large amount of clinically meaningful data by adding the workflow of the present invention to a workflow inside an existing medical institution.

Meanwhile, in general, to treat a COPD patient, COPD analysis is performed using CT data. However, there are many cases where COPD analysis is not performed on a CT image not taken for the purpose of diagnosing COPD even when it contains sufficient anatomical information to perform COPD analysis. The medical image analysis method performed by the server 110 of the present invention automatically searches for and analyzes medical images when target images concern chest CT and a predetermined slice condition is satisfied, thereby supporting the diagnosis of hidden COPD. Due to this, the data stored in the second database 140 also become abundant, and thus a big data database may be built.

Furthermore, among conventional attempts to expand a patient's medical information, there are cases of extracting the patient's diagnostic information without considering the patient's privacy. In contrast, the medical image analysis method of the present invention targets only patients who have referred to a medical institution for the diagnosis of a specific disease. Accordingly, the present invention may be viewed as an invention that can be easily adopted in the medical field as a method for securing a large amount of clinically meaningful data in that it may be possible to additionally discover a disease, not discovered by a patient or a medical staff, without invading a patient's privacy.

The image processing step of detecting a patient's disease and the step of extracting a region of interest may be performed by a computer-aided diagnosis (CAD) module. The CAD module may be a rule-based solution, or a solution based on artificial neural network-based machine learning.

The setting of a retrieve condition may be efficiently adjusted by learning or training. For example, a conventional COPD diagnostic module can be applied only to a chest CT image including 200 or more slices. However, when an improved COPD diagnostic module lowers the number of required slices, a retrieve condition may be adjusted based on the number of required slices lowered by learning or training.

Unlike the related arts for extracting unnecessarily many patterns from limited data, the present invention focuses on an increase in the amount of data and the extraction of clinically meaningful patterns, so that the clinical reliability of extracted information is high and the extracted information may be utilized in medical institutions in various ways.

The medical image analysis method according to an embodiment of the present invention may be implemented in the form of program instructions executable by a variety of computer means, and may be stored in a computer-readable storage medium. The computer-readable storage medium may include program instructions, a data file, and a data structure solely or in combination. The program instructions which are stored in the medium may be designed and constructed particularly for the present invention, or may be well known and available to those skilled in the field of computer software. Examples of the computer-readable storage medium include magnetic media such as a hard disk, a floppy disk and a magnetic tape, optical media such as CD-ROM and a DVD, magneto-optical media such as a floptical disk, and hardware devices particularly configured to store and execute program instructions such as ROM, RAM, and flash memory. Examples of the program instructions include not only machine language code that is constructed by a compiler but also high-level language code that can be executed by a computer using an interpreter or the like. The above-described hardware components may be configured to act as one or more software modules that perform the operation of the present invention, and vice versa.

However, the present invention is not limited and restricted to the embodiments. Throughout the drawings, the same reference symbols denote the same members. The lengths, heights, sizes, widths, etc. introduced in the embodiments and drawings of the present invention may be exaggerated to help an understanding of the present invention.

According to the present invention, a large amount of clinically meaningful data may be generated by adding the workflow of the present invention to a workflow for processing medical information inside a medical institution. In this case, according to the present invention, a large amount of clinically meaningful data may be generated based on medical information inside the medical institution without invading patients' privacy.

According to the present invention, a medical information database may be enriched and a big data database may be built, the process of which may be performed in a server-based environment.

According to the medical image analysis technique of the present invention, retrieve similar cases may be effectively supported and more cases and a larger amount of information may be presented when similar cases are searched for.

According to the medical image analysis technique of the present invention, a medical image is automatically searched for in the first database, a first feature for a first medical image is quantified, and the first feature is stored in the second database in association with the first medical image and a retrieve condition, thereby generating statistical information including the first feature in connection with the retrieve condition.

According to the medical image analysis technique of the present invention, there may be provided a means for, when a second medical image, which is a new medical image of a patient, is received, quantifying a second feature for the second medical image and comparing the second feature with statistical information, thereby quantifying and evaluating the severity of a patient's symptoms shown in the second medical image.

According to the medical image analysis technique of the present invention, there may be provided a quantitative retrieve condition for retrieve similar cases having a quantitative feature similar to that of the second medical image, and the efficiency of retrieve similar cases may be increased.

According to the medical image analysis technique of the present invention, even when there is no reference image of a patient for comparison, there may be provided a user menu adapted to add a quantitative retrieve condition for a quantified feature to a retrieve condition, and a third medical image satisfying a certain quantitative retrieve condition may be provided to a user as a search result. In this case, when combined with statistical information, it may be possible to retrieve cases based on the severity of a specific disease according to the quantity retrieve condition, thereby retrieving typical cases according to their severity and then generating reference images required for training for clinicians and radiologists and training for machine learning based on an artificial neural network.

Moreover, according to the present invention, results obtained through comparison with statistical information may be generated as a diagnostic report or label information for each medical image. When the label information is added to each medical image, a reference image required for training for machine learning based on an artificial neural network may be generated.

The present invention was derived from the research conducted as a part of the Innovative Enterprise Technology Development Project sponsored by the Korea Technology and Information Promotion Agency for SMEs of the Korean Ministry of SMEs and Startups (MSS) [Project Management Number: 52464035; and Project Name: Development of Software for Fully Automated Analysis of Chronic Obstructive Pulmonary Disease (COPD) using Artificial Intelligence and Retrieve Similar Cases].

While the present invention has been described in conjunction with specific details, such as specific components, and limited embodiments and diagrams above, these are provided merely to help an overall understanding of the present invention. The present invention is not limited to these embodiments, and various modifications and alterations may be made based on the foregoing description by those having ordinary knowledge in the art to which the present invention pertains.

Therefore, the technical spirit of the present invention should not be determined based only on the described embodiments, and not only the following claims but also all equivalents to the claims and equivalent modifications should be construed as falling within the scope of the spirit of the present invention.

What is claimed is:

1. A medical image analysis method performed by a server, the medical image analysis method comprising:
automatically retrieving a medical image satisfying a retrieve condition in a first database in which medical images are stored, and receiving a retrieved image as a first medical image;
performing image processing on the first medical image, and extracting at least one first region of interest from the first medical image;
quantifying a first feature extracted for the first medical image and the at least one first region of interest; and
storing the first feature in a second database in association with the first medical image and the retrieve condition.

2. The medical image analysis method of claim 1, further comprising:
incorporating the first feature into a data set related to the retrieve condition;
generating statistical information about the data set related to the retrieve condition; and
storing the statistical information in the second database in association with the retrieve condition.

3. The medical image analysis method of claim 2, further comprising:
receiving a new medical image of a patient as a second medical image;
performing image processing on the second medical image, and extracting at least one second region of interest from the second medical image;
quantifying a second feature extracted for the second medical image and the at least one second region of interest; and
generating results, obtained by comparing the second feature with the statistical information, as a diagnostic report for the second medical image.

4. The medical image analysis method of claim 1, further comprising:
providing a user with a user menu adapted to add a quantitative retrieve condition for a quantified feature to the retrieve condition;
determining the quantitative retrieve condition based on the user's input via the user menu; and
retrieving a medical image satisfying the quantitative retrieve condition and the retrieve condition in the first database, and providing a retrieved image to the user as a third medical image.

5. The medical image analysis method of claim 3, further comprising:
generating a quantitative retrieve condition for a quantified feature based on the second feature; and
retrieving a medical image satisfying the quantitative retrieve condition and the retrieve condition in the first database, and providing a retrieved image to the user as a fourth medical image.

6. The medical image analysis method of claim 2, further comprising:
generating results, obtained by comparing the first feature with the statistical information, as a diagnostic report for the first medical image.

7. The medical image analysis method of claim 6, further comprising:
generating label information for the first medical image based on the diagnostic report for the first medical image; and
generating a fifth medical image by adding the label information to the first medical image.

8. The medical image analysis method of claim 1, wherein a type of the at least one first region of interest, a category of the first feature, and an image processing process for the first medical image are determined based on the retrieve condition in advance.

9. The medical image analysis method of claim 1, wherein the retrieve condition is predetermined to include at least one of a type and feature of the at least one first region of interest, a category of the first feature, and an image processing process for the first medical image in a common fashion.

10. A medical image analysis server comprising at least one processor, wherein the at least one processor is configured to:
automatically transmit a retrieval query to a first database in which medical images are stored;
control a receiving interface so that the receiving interface receives a first medical image satisfying the retrieval query from the first database;
perform image processing on the first medical image and extract at least one first region of interest from the first medical image;
quantify a first feature extracted for the first medical image and the at least one first region of interest; and
store the first feature in a second database in association with the first medical image and the retrieve condition.

11. The medical image analysis server of claim 10, wherein the at least one processor is further configured to:
incorporate the first feature into a data set related to the retrieve condition;
generate statistical information about the data set related to the retrieve condition; and
store the statistical information in the second database in association with the retrieve condition.

12. The medical image analysis server of claim 11, wherein the at least one processor is further configured to:
receive a new medical image of a patient as a second medical image;
perform image processing on the second medical image, and extracting at least one second region of interest from the second medical image;
quantify a second feature extracted for the second medical image and the at least one second region of interest; and
generate results, obtained by comparing the second feature with the statistical information, as a diagnostic report for the second medical image.

13. The medical image analysis server of claim 12, wherein the at least one processor is further configured to:
generate a quantitative retrieve condition for a quantified feature based on the second feature; and
retrieve a medical image satisfying the quantitative retrieve condition and the retrieve condition in the first database and provide a retrieved image to a user as a fourth medical image.
